(19) United States
(12) Patent Application Publication
(10) Pub. No.: US 2026/0207221 A1
(43) Pub. Date: Jul. 23, 2026

(54) Ultrasound Systems, Probes and Methods Thereof for Needle Tracking and Guidance

(71) Applicant: Bard Access Systems, Inc., Salt Lake City, UT (US)

(72) Inventor: Shayne Messerly, Kaysville, UT (US)

(21) Appl. No.: 19/445,231

(22) Filed: Jan. 9, 2026

Related U.S. Application Data
(60) Provisional application No. 63/748,859, filed on Jan. 23, 2025.

(51) Int. Cl. A61B 17/34; A61B 8/00; A61B 17/00; A61B 34/20

(52) U.S. Cl. CPC A61B 17/3403; A61B 8/4488; A61B 34/20; A61B 2017/00199; A61B 2017/3413; A61B 2034/2063; A61B 2034/2065

(57) ABSTRACT

Ultrasound systems, probes, and methods provide the accuracy of tracking a needle's trajectory with in-plane visualization and the optimal views of anatomical structures associated with out-of-plane visualization. Such an ultrasound system for needle tracking and guidance can include an ultrasound probe having an imaging array and a linear tracking array perpendicular to the imaging array. A console of the ultrasound system can include memory having executable instructions that instantiate system processes for imaging with the imaging array as well as needle tracking with the tracking array when executed by one or more processors. An image-generating process generates ultrasound images of a target area or anatomical structure thereof from echoed ultrasound signals corresponding to the patient. A needle-tracking process generates needle-tracking data from the echoed ultrasound signals corresponding to a needle. A needle-guiding process provides on-screen guidance of the needle to the target area or anatomical structure thereof.

### PRIORITY

This application claims the benefit of priority to U.S. Provisional Application No. 63/748,859, filed Jan. 23, 2025, which is incorporated by reference in its entirety into this application.

### BACKGROUND

Ultrasound guidance enhances safety and accuracy in procedures involving percutaneous needle punctures by allowing real-time visualization of both the needles and the anatomical structures being accessed (or avoided) by such needles when inserted into patients.

Two techniques are generally used for visualizing needle insertion under ultrasound: in-plane (or long-axis) and out-of-plane (or short axis) visualization. With in-plane visualization, a needle is aligned with an ultrasound beam. This provides a longitudinal view of the needle in resulting ultrasound images, which facilitates accurate tracking the needle's trajectory during insertion thereof. With out-of-plane visualization, the needle is perpendicular to the ultrasound beam. This provides a cross-sectional view of the needle in the resulting ultrasound images, wherein the needle appears as a dot. Tracking the needle's trajectory can be more challenging during insertion with out-of-plane visualization.

Despite the foregoing needle-tracking challenge, percutaneous needle punctures with out-of-plane visualization under ultrasound are often preferred. This preference arises because out-of-plane visualization can provide a more optimal view of target and surrounding anatomical structures. Additionally, out-of-plane needle approaches to the target anatomical structures can offer more flexibility in needle angulation and entry points, which accommodate different anatomical variations and patient positioning.

What is needed is ultrasound guidance that provides the accuracy of tracking a needle's trajectory with in-plane visualization and the optimal views of anatomical structures associated with out-of-plane visualization. Disclosed herein are ultrasound systems, probes, and methods thereof that address the foregoing.

### SUMMARY

Disclosed herein is an ultrasound system for needle tracking and guidance. The ultrasound system includes, in some embodiments, an ultrasound probe and a console. The ultrasound probe includes an imaging array and a linear tracking array perpendicular to the imaging array. The imaging array includes a plurality of ultrasound-transducing elements disposed in a probe head of the ultrasound probe. The tracking array includes two or more ultrasound-transducing elements. The ultrasound-transducing elements of each array of the imaging array and the tracking array are oriented to emit source ultrasound signals into a patient and transduce echoed ultrasound signals from both the patient and a needle inserted into the patient. The console contains electronic components and circuitry including memory and one or more processors. The memory includes executable instructions that instantiate one or more system processes for imaging with the imaging array as well as needle tracking with the tracking array when the executable instructions are executed by the processor(s). The system processes include an image-generating process, a needle-tracking process, and a needle-guiding process. The image-generating process generates ultrasound images of a target area or anatomical structure thereof from the echoed ultrasound signals corresponding to the patient. The needle-tracking process generates needle-tracking data from the echoed ultrasound signals corresponding to the needle. The needle-guiding process provides on-screen guidance of the needle to the target area or anatomical structure thereof.

In some embodiments, the tracking array is flush with or recessed into either the probe head or a protrusion laterally extending from the probe head so as to not distally extend past the imaging array.

In some embodiments, the tracking array is angled into the imaging array.

In some embodiments, the ultrasound probe includes a marker on a same side of the ultrasound probe as that including the tracking array. The marker indicates the needle should be inserted into the patient on an opposite side of the ultrasound probe from the marker.

In some embodiments, the ultrasound probe includes a marker on an opposite side of the ultrasound probe than that including the tracking array. The marker indicates the needle should be inserted into the patient on a same side of the ultrasound probe as the marker.

In some embodiments, the ultrasound probe includes a needle-guide mount extending from an opposite side of the ultrasound probe than that including the tracking array.

In some embodiments, the ultrasound system is configured for both A-mode and B-mode pulsed-wave (“PW”) ultrasound.

In some embodiments, the needle-tracking process utilizes the tracking array for A-mode PW ultrasound.

In some embodiments, ultrasound-transducing elements of the tracking array simultaneously emit the source ultrasound signals and transduce the echoed ultrasound signals for simultaneous depth determinations along different points of the needle.

In some embodiments, the needle-guiding process utilizes the depth determinations along the different points of the needle to render a guidance overlay over the ultrasound images for the on-screen guidance of the needle to the target area or anatomical structure thereof.

In some embodiments, the source ultrasound signals emitted by the ultrasound-transducing elements of the tracking array are frequency modulated. Each ultrasound-transducing element of the tracking array emits its corresponding source ultrasound signals at a unique frequency for independently processing the echoed ultrasound signals transduced by each ultrasound-transducing element of the tracking array in accordance with the unique frequency thereof.

In some embodiments, the source ultrasound signals emitted by the ultrasound-transducing elements of the tracking array are phase modulated. Each ultrasound-transducing element of the tracking array emits its corresponding source ultrasound signals with a unique phase shift for independently processing the echoed ultrasound signals transduced by each ultrasound-transducing element of the tracking array in accordance with the unique phase shift thereof.

In some embodiments, the source ultrasound signals emitted by the ultrasound-transducing elements of the tracking array are pulse encoded. Each ultrasound-transducing element of the tracking array emits its corresponding source ultrasound signals with a unique pulse train for independently processing the echoed ultrasound signals transduced by each ultrasound-transducing element of the tracking array in accordance with the unique pulse train thereof.

In some embodiments, processing the echoed ultrasound signals transduced by each ultrasound-transducing element of the tracking array includes amplitude gating to filter out the echoed ultrasound signals from the patient and isolate the echoed ultrasound signals from the needle. The echoed ultrasound signals from the needle have much greater amplitudes than the echoed ultrasound signals from the patient.

In some embodiments, the image-generating process utilizes the imaging array for B-mode PW ultrasound.

In some embodiments, the source ultrasound signals emitted by the ultrasound-transducing elements of the tracking array being modulated or encoded differentiates them from the source ultrasound signals emitted by the ultrasound-transducing elements of the imaging array and, thus, the echoed ultrasound signals transduced by the ultrasound-transducing elements of the tracking array and the imaging array.

Also disclosed is an ultrasound probe for needle tracking and guidance. The ultrasound probe includes, in some embodiments, an imaging array and a linear tracking array perpendicular to the imaging array. The imaging array includes a plurality of ultrasound-transducing elements disposed in a probe head of the ultrasound probe. The tracking array includes two or more ultrasound-transducing elements. The ultrasound-transducing elements of each array of the imaging array and the tracking array are oriented to emit source ultrasound signals into a patient and transduce echoed ultrasound signals from both the patient and a needle inserted into the patient.

In some embodiments, the tracking array is flush with or recessed into either the probe head or a protrusion laterally extending from the probe head so as to not distally extend past the imaging array.

In some embodiments, the tracking array is angled into the imaging array.

In some embodiments, the ultrasound probe includes a marker on a same side of the ultrasound probe as that including the tracking array. The marker indicates the needle should be inserted into the patient on an opposite side of the ultrasound probe from the marker.

In some embodiments, the ultrasound probe includes a marker on an opposite side of the ultrasound probe than that including the tracking array. The marker indicates the needle should be inserted into the patient on a same side of the ultrasound probe as the marker.

In some embodiments, the ultrasound probe includes a needle-guide mount extending from an opposite side of the ultrasound probe than that including the tracking array.

Also disclosed herein is a method of an ultrasound system for needle tracking and guidance. The method includes, in some embodiments, instantiating one or more system processes for imaging and needle tracking with an ultrasound probe when executable instructions in memory of a console of the ultrasound system are executed by one or more processors of the console. The method also includes generating ultrasound images of a target area or anatomical structure of a patient with an image-generating process of the system process(es). The ultrasound images are generated from source ultrasound signals emitted from an imaging array of a plurality of ultrasound-transducing elements disposed in a probe head of the ultrasound probe and echoed ultrasound signals from the patient transduced by the ultrasound-transducing elements of the imaging array. The method also includes generating needle-tracking data of a needle approaching the target area or anatomical structure of the patient with a needle-tracking process of the system process(es). The needle-tracking data are generated from source ultrasound signals emitted from a tracking array of two or more ultrasound-transducing elements perpendicular to the imaging array and echoed ultrasound signals from the needle transduced by the ultrasound-transducing elements of the tracking array. The method also includes providing on-screen guidance of the needle to the target area or anatomical structure thereof with a needle-guiding process of the system process(es).

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates an ultrasound system for needle tracking and guidance in accordance with some embodiments.

FIG. 2 illustrates a block diagram of the ultrasound system in accordance with some embodiments.

FIG. 3 illustrates a detailed side view of an ultrasound probe of the ultrasound system in accordance with some embodiments.

FIG. 4 illustrates a detailed side view of the ultrasound probe in accordance with some other embodiments.

FIG. 5 illustrates an end-on view of a distal end of the ultrasound probe in accordance with some embodiments.

FIG. 6 provides a schematic illustrating the ultrasound probe in use during a percutaneous needle puncture to establish vascular access in accordance with some embodiments.

FIG. 7 illustrates the ultrasound system in use during a percutaneous needle puncture but prior to establishing vascular access in accordance with some embodiments.

FIG. 8 illustrates the ultrasound system in use during a percutaneous needle puncture at a time of establishing vascular access in accordance with some embodiments.

FIG. 9 illustrates on-screen guidance of a needle to a target blood vessel in accordance with some embodiments.

FIG. 10 illustrates continued on-screen guidance of the needle to the target blood vessel in accordance with some embodiments.

FIG. 11 illustrates final on-screen guidance of the needle to the target blood vessel in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

“Proximal” is used to indicate a portion, section, piece, element, or the like of a medical device intended to be near or relatively nearer to a clinician when the medical device is used on a patient. For example, a “proximal portion” or “proximal section” of the medical device includes a portion or section of the medical device intended to be near the clinician when the medical device is used on the patient. Likewise, a “proximal length” of the medical device includes a length of the medical device intended to be near the clinician when the medical device is used on the patient. A “proximal end” of the medical device is an end of the medical device intended to be near the clinician when the medical device is used on the patient. The proximal portion, the proximal section, or the proximal length of the medical device need not include the proximal end of the medical device. Indeed, the proximal portion, the proximal section, or the proximal length of the medical device can be short of the proximal end of the medical device. However, the proximal portion, the proximal section, or the proximal length of the medical device can include the proximal end of the medical device. Should context not suggest the proximal portion, the proximal section, or the proximal length of the medical device includes the proximal end of the medical device, or if it is deemed expedient in the following description, “proximal portion,” “proximal section,” or “proximal length” can be modified to indicate such a portion, section, or length includes an end portion, an end section, or an end length of the medical device for a “proximal end portion,” a “proximal end section,” or a “proximal end length” of the medical device, respectively.

“Distal” is used to indicate a portion, section, piece, element, or the like of a medical device intended to be near, relatively nearer, or even in a patient when the medical device is used on the patient. For example, a “distal portion” or “distal section” of the medical device includes a portion or section of the medical device intended to be near, relatively nearer, or even in the patient when the medical device is used on the patient. Likewise, a “distal length” of the medical device includes a length of the medical device intended to be near, relatively nearer, or even in the patient when the medical device is used on the patient. A “distal end” of the medical device is an end of the medical device intended to be near, relatively nearer, or even in the patient when the medical device is used on the patient. The distal portion, the distal section, or the distal length of the medical device need not include the distal end of the medical device. Indeed, the distal portion, the distal section, or the distal length of the medical device can be short of the distal end of the medical device. However, the distal portion, the distal section, or the distal length of the medical device can include the distal end of the medical device. Should context not suggest the distal portion, the distal section, or the distal length of the medical device includes the distal end of the medical device, or if it is deemed expedient in the following description, “distal portion,” “distal section,” or “distal length” can be modified to indicate such a portion, section, or length includes an end portion, an end section, or an end length of the medical device for a “distal end portion,” a “distal end section,” or a “distal end length” of the medical device, respectively.

“Logic” can be hardware, firmware, or software configured to perform one or more functions. As hardware, logic can include circuitry having data processing functionality, data storage functionality, or both. An example of such circuitry can include, but is not limited to, a hardware processor (e.g., a microprocessor, one or more processor cores, a digital-signal processor [“DSP”], a programmable gate array [“PGA”], a microcontroller, an application-specific integrated circuit [“ASIC”], etc.) or semiconductor memory. As firmware, the logic can be stored in persistent storage. As software, logic can include one or more processes, instances, Application Programming Interfaces (“APIs”), subroutines, functions, applets, servlets, or routines. Logic can also include source code, object code, a shared library, a dynamic link library (“DLL”), or even one or more instructions. Such software can be stored in any type of suitable non-transitory storage medium or transitory storage medium (e.g., electrical, optical, acoustical, or any other form of propagated signal including carrier waves, infrared signals, or digital signals). An example of a non-transitory storage medium can include, but is not limited to, a programmable circuit; non-persistent storage such as volatile memory (e.g., any type of random-access memory [“RAM”]); or persistent storage such as non-volatile memory (e.g., read-only memory [“ROM”], power-backed RAM, flash memory, phase-change memory, etc.), a solid-state drive, a hard-disk drive, an optical-disc drive, or a portable memory device.

Unless defined otherwise, all technical and scientific terms used herein have the same meaning as commonly understood by those of ordinary skill in the art.

### Ultrasound System

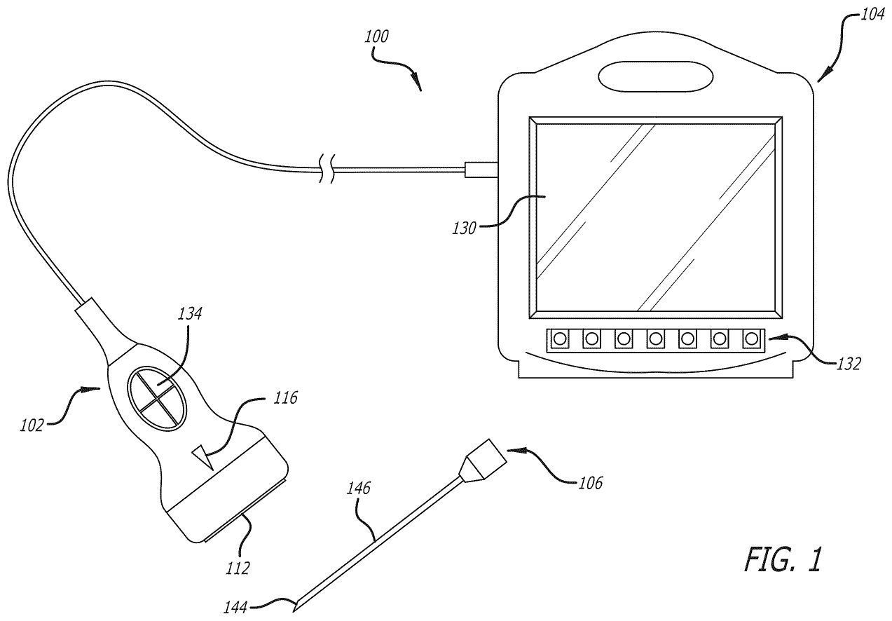

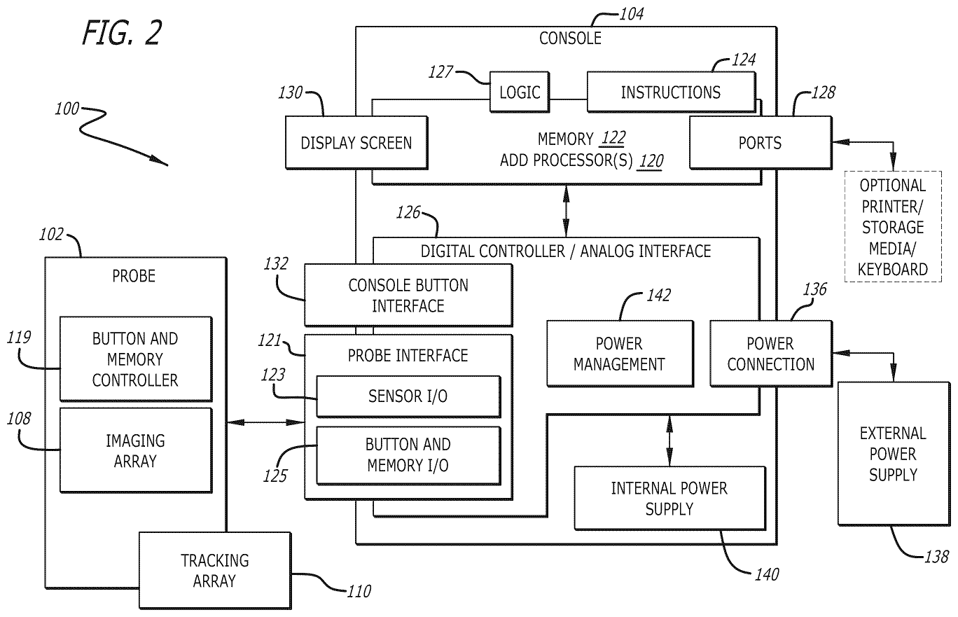

FIG. 1 illustrates an ultrasound system 100 for needle tracking and guidance in accordance with some embodiments. FIG. 2 illustrates a block diagram of the ultrasound system 100 in accordance with some embodiments.

As shown, the ultrasound system 100 includes, in some embodiments, an ultrasound probe 102 and a console 104, each of which is described in further detail below. Notably, the ultrasound system 100 can further include a needle 106, as shown, particularly if the needle 106 is configured with echogenic features or the like for enhanced needle tracking with the ultrasound system 100.

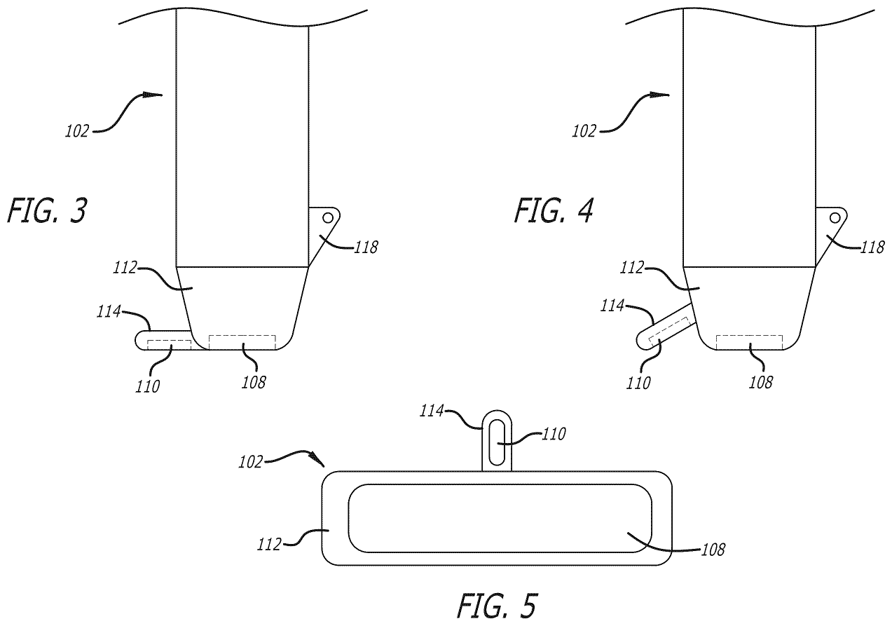

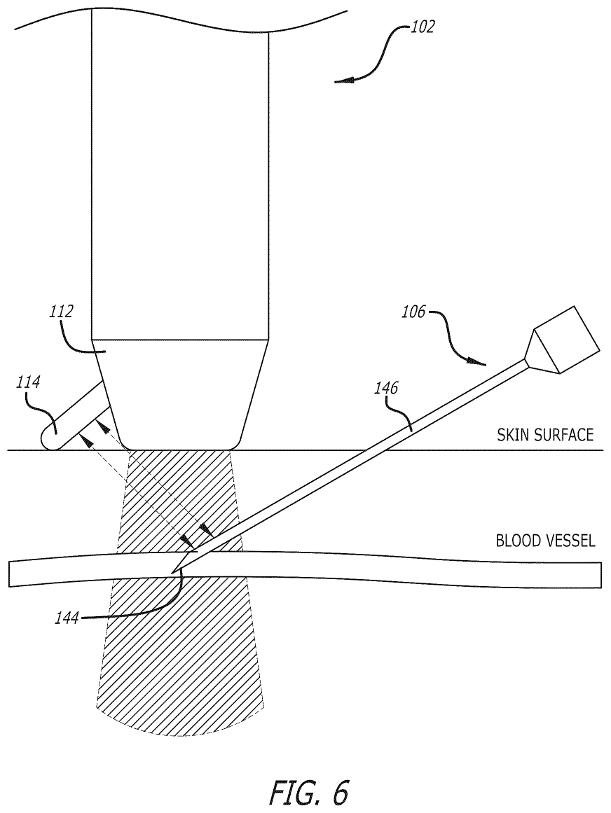

FIGS. 3 and 4 illustrate detailed side views of the ultrasound probe 102 in accordance with some embodiments. FIG. 5 illustrates an end-on view of a distal end of the ultrasound probe 102 in accordance with some other embodiments. And FIG. 6 provides a schematic illustrating the ultrasound probe 102 in use during a percutaneous needle puncture to establish vascular access in accordance with some embodiments.

As best shown in FIG. 5, the ultrasound probe 102 includes an imaging array 108 and a linear tracking array 110 perpendicular to the imaging array 108; however, it should be understood that a single ultrasound-transducing element or, namely, a tracking element, configured like those of the tracking array 110 can be used as an alternative to the tracking array 110 in some embodiments. The ultrasound-transducing elements of each array of the imaging array 108 and the tracking array 110 are oriented to emit source ultrasound signals into a patient and transduce echoed ultrasound signals from both the patient and a needle inserted into the patient. Configured as such, the ultrasound probe 102 and the ultrasound system 100 of which it is part provide optimal views of anatomical structures associated with out-of-plane visualization via the imaging array 108 and accurate needle tracking with in-plane visualization via the tracking array 110.

The imaging array 108 includes a plurality of ultrasound-transducing elements disposed in a probe head 112 of the ultrasound probe 102. Such an imaging array 108 and the ultrasound-transducing elements thereof are useful for, but not limited to, B-mode pulsed-wave (“PW”) ultrasound.

The tracking array 110 includes two or more ultrasound-transducing elements useful for, but not limited to, A-mode PW or continuous-wave (“CW”) ultrasound, the latter A-mode CW ultrasound having a chirped or frequency-swept signal. The tracking array 110 can be flush with or recessed into the probe head 112. Alternatively, the tracking array 110 can be flush with or recessed into a protrusion 114 laterally extending from the probe head 112. When recessed into the probe head 112 or the protrusion 114, the tracking array 110 or individual ultrasound-transducing elements thereof can be angled into the imaging array 108 so as to be directed toward the needle 106 when the needle 106 is used on an opposite side of the ultrasound probe 102 from the tracking array 110, for example, during a percutaneous puncture as shown in FIG. 6. Angling of the tracking array 110 is conveniently shown in FIGS. 4 and 6 by way of the protrusion 114, itself, being angled, which represents yet another embodiment of the ultrasound probe 102. Regardless of how the tracking array 110 is incorporated into the ultrasound probe 102, it is preferable for patient comfort that the tracking array 110 or any component of which it is part (e.g., the protrusion 114) not distally extend past the imaging array 108.

The ultrasound probe 102 can include a marker 116 on a side of the ultrasound probe 102 or the probe head 112 on that side of the ultrasound probe 102. Indeed, the marker 116 can be on a same side of the ultrasound probe 102 as that including the tracking array 110, or the marker 116 can be on an opposite side of the ultrasound probe 102 than that including the tracking array 110. In an example, if the tracking array 110 is concealed in the probe head 112 instead of the conspicuous protrusion 114, the marker 116 can be on the same side of the ultrasound probe 102 as that including the tracking array 110, thereby indicating the side of the ultrasound probe 102 including the tracking array 110. Accordingly, such a marker 116 also indicates the needle 106 should be inserted into the patient on the opposite side of the ultrasound probe 102 from the marker 116. Whether the tracking array 110 is concealed in the probe head 112 or more conspicuously disposed in the protrusion 114, the marker 116 can be on the opposite side of the ultrasound probe 102 than that including the tracking array 110, thereby indicating the needle 106 should be inserted into the patient on the same side of the ultrasound probe 102 as the marker 116.

Additionally or alternatively, the ultrasound probe 102 can include a needle-guide mount 118 configured for coupling a needle guide thereto for guiding the needle 106 at a proper angle (e.g., approach angle) under the probe head 112 such that the needle 106 intersects with the imaging plane established by the imaging array 108. When present, as in FIGS. 3 and 4, the needle-guide mount 118 extends from the opposite side of the ultrasound probe 102 than that including the tracking array 110. In this way, the needle-guide mount 118 can alternatively or additionally function as a marker to indicate the side of the ultrasound probe 102 from which the needle 106 should be inserted into the patient.

Adverting to FIGS. 1 and 2, the ultrasound probe 102 can further include a button-and-memory controller 119 for governing operation of the ultrasound probe 102 and control buttons 134 thereof. The button-and-memory controller 119 can include non-volatile memory such as electrically erasable, programmable, read-only memory (“EEPROM”). When the ultrasound probe 102 is operably connected to the console, the button-and-memory controller 119 can be in operable communication with a probe interface 121 of the console; however, the ultrasound probe 102 can include a wireless communications module (not shown) to wirelessly communicate with the console and its wireless communications module (not shown) as opposed to over the probe interface. Regardless, the probe interface 121 of the console can include an ultrasound-sensor input-output (“I/O”) component 123 for operably communicating with the imaging and tracking arrays 108 and 110 of the ultrasound probe 102 as well as a button-and-memory I/O component 125 for operably communicating with the button-and-memory controller 119 of the ultrasound probe 102.

The console 104 contains electronic circuitry and components including one or more processors 120, memory 122 (e.g., EEPROM) including executable instructions 124, and logic 127 for instantiating and running one or more system processes. The processor(s) 120, the memory 122 including the instructions 124 stored therein, and the logic 127 of the console 104 can be configured for controlling various functions of the ultrasound system 100 including, but not limited to, imaging with the imaging array 108 as well as needle tracking with the tracking array 110. Further, the console 104 can include a digital controller or analog interface 126 in operable communication with the processor(s) 120, the memory 122, the logic 127 and any one or more other components of the ultrasound system 100, for example, the ultrasound probe 102, to govern operation between them.

The console 104 can also include ports 128 for operably connecting additional or optional components of the ultrasound system 100 including peripheral devices including standalone monitors, storage media, printers, or the like. The ports 128 can be universal serial bus (“USB”) ports of any kind; however, ports other than USB ports can be incorporated into the console 104. In an example, the ports 128 can include a DisplayPort (“DP”) port or a high-definition multimedia interface (“HDMI”) port for operably connecting a standalone monitor if the display screen 130 is separate from the console 104.

The console 104 can also include a display screen 130 such as a liquid crystal display (“LCD”) screen integrated into the console 104 to display information to a clinician before, during, or after establishing vascular access with the ultrasound system 100. For example, the display screen 130 can be used to display the ultrasound image of the target area of the patient attained by the ultrasound probe 102. Alternatively, the display screen 130 can be separate from the console 104 such as in the standalone monitor set forth above instead of integrated into the console 104. Notably, the console 104 can also include a console button interface 132. In combination with the control buttons 134 on the ultrasound probe 102, the console button interface 132 of the console 104 can be used by the clinician to immediately call up a desired mode of the ultrasound system 100 on the display for use by the clinician.

Lastly, the console 104 can also include a power connection 136 to enable an operable connection of the console 104 to an external power supply 138. The console 104 can also include an internal power supply 140 (e.g., disposable or rechargeable battery) together with the external power supply 138 or exclusive of the external power supply 138. Power management logic 142 with the digital controller or analog interface 126 of the console 104 can regulate power use and distribution within the console 104 as well as at least some of the additional or optional components of the ultrasound system 100 or when such components are operably connected to the console 104.

The system process(es) can include one or more processes selected from at least an image-generating process, a needle-tracking process, and a needle-guiding process. Notably, the image-generating process utilizes the imaging array 108 for B-mode PW ultrasound, and the needle-tracking process can utilize the tracking array 110 or tracking element for A-mode PW ultrasound.

The image-generating process generates ultrasound images of a target area or anatomical structure thereof from the echoed ultrasound signals corresponding to the patient in accordance with their time of flight and intensity, which intensity is proportional to the square of the amplitude. Notably, processing the echoed ultrasound signals transduced by each ultrasound-transducing element of the imaging array 108 can include amplitude gating to filter out the echoed ultrasound signals from the needle 106 and isolate the echoed ultrasound signals from the patient. Indeed, on account of acoustic impedance mismatches at the boundaries between the needle 106 and various tissues of the patient, the echoed ultrasound signals from the patient have much smaller amplitudes than the echoed ultrasound signals from the needle 106 allowing the echoed ultrasound signals from the needle 106 to be filtered out by amplitude gating.

The needle-tracking process generates needle-tracking data from the echoed ultrasound signals corresponding to the needle 106 in accordance with their time of flight and amplitude. Like that set forth above, processing the echoed ultrasound signals transduced by each ultrasound-transducing element of the tracking array 110 can include amplitude gating to filter out the echoed ultrasound signals from the patient and isolate the echoed ultrasound signals from the needle 106. Again, on account of acoustic impedance mismatches at the boundaries between the needle 106 and various tissues of the patient, the echoed ultrasound signals from the needle 106 have much greater amplitudes than the echoed ultrasound signals from the patient allowing the echoed ultrasound signals from the patient to be filtered out by amplitude gating.

With or without the foregoing amplitude gating, the source ultrasound signals emitted by the ultrasound-transducing elements of the tracking array 110 can be modulated or encoded like that set forth below to differentiate them from the source ultrasound signals emitted by the ultrasound-transducing elements of the imaging array 108. Thus, the echoed ultrasound signals transduced by the ultrasound-transducing elements of the tracking array 110 and the imaging array 108 are likewise differentiated. Such differentiation allows differential signal processing by the image-generating and needle-guiding processes, which is beneficial when the ultrasound-transducing elements of the imaging and tracking arrays 108 and 110 simultaneously emit the source ultrasound signals and transduce the echoed ultrasound signals. However, it should be understood that, in some embodiments, the ultrasound-transducing elements of the imaging and tracking arrays 108 and 110 alternately emit the source ultrasound signals and transduce the echoed ultrasound signals, thereby obviating any need for modulating or encoding the source ultrasound signals emitted by the tracking array 110 for differential signal processing unless desired.

Notwithstanding, any modulating or encoding of the source ultrasound signals emitted by the tracking array 110 to differentiate them from the source ultrasound signals emitted by the imaging array 108, the modulating and encoding can additionally or alternatively be among the ultrasound-transducing elements of the tracking array 110 themselves for differential signal processing. This is beneficial when the ultrasound-transducing elements of the tracking array 110 simultaneously emit the source ultrasound signals and transduce the echoed ultrasound signals for simultaneous depth determinations along different points of the needle 106. Depths determined for different points along the needle 106 at any given time, which include the depths for a needle tip 144 and one or more points along a needle shaft 146, constitute at least a portion of the needle-tracking data for the needle-guiding process the guidance overlay 148 rendered thereby over the ultrasound images.

In an example of modulating or encoding the source ultrasound signals emitted by the tracking array 110, the source ultrasound signals can be frequency modulated. When each ultrasound-transducing element of the tracking array 110 is independently frequency modulated, the ultrasound-transducing element emits its corresponding source ultrasound signals at a unique frequency for independently processing the echoed ultrasound signals transduced by each ultrasound-transducing element of the tracking array 110 in accordance with the unique frequency thereof.

In another example of modulating or encoding the source ultrasound signals emitted by the tracking array 110, the source ultrasound signals can be phase modulated. When each ultrasound-transducing element of the tracking array 110 is independently phased modulated, the ultrasound-transducing element emits its corresponding source ultrasound signals with a unique phase shift (e.g., 0°, 90°, 180°, 270°) for independently processing the echoed ultrasound signals transduced by each ultrasound-transducing element of the tracking array 110 in accordance with the unique phase shift thereof.

In another example of modulating or encoding the source ultrasound signals emitted by the tracking array 110, the source ultrasound signals can be pulse encoded. When each ultrasound-transducing element of the tracking array 110 is independently pulse encoded, the ultrasound-transducing element emits its corresponding source ultrasound signals with a unique pulse train for independently processing the echoed ultrasound signals transduced by each ultrasound-transducing element of the tracking array 110 in accordance with the unique pulse train thereof. Such a pulse train can be unique with respect to any one or more pulse characteristics selected from pulse amplitude, width, spacing, and repetition frequency including any modulations of the selected pulse characteristic(s).

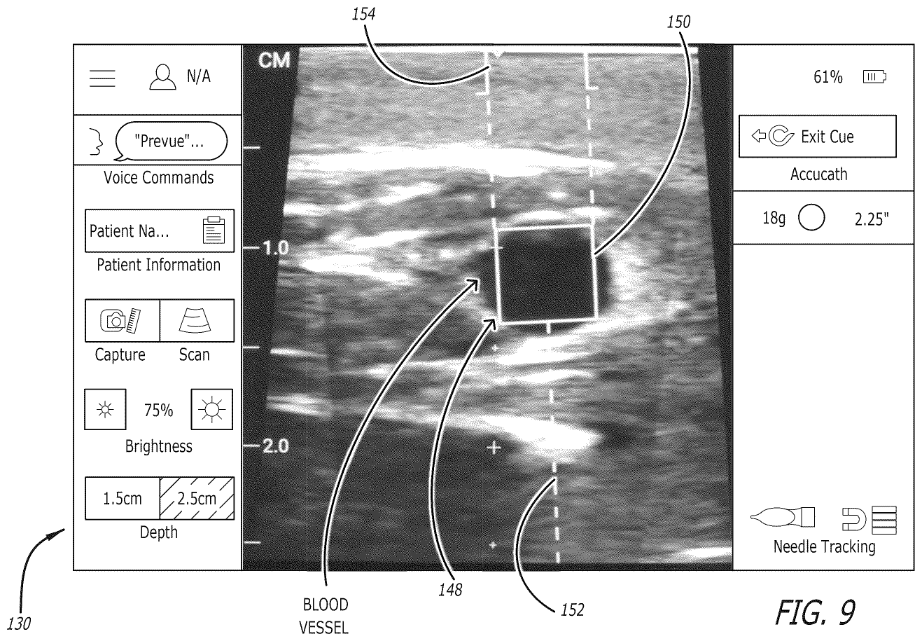

FIGS. 9-11 illustrate on-screen guidance of the needle 106 to a target area or anatomical structure thereof in accordance with some embodiments.

As shown, the needle-guiding process can provide the on-screen guidance of the needle 106 to at least a target blood vessel as the target anatomical structure. As set forth above, the needle-tracking process generates the needle-tracking data from the echoed ultrasound signals corresponding to the needle 106 in accordance with their time of flight and amplitude, from which the depths along the different points (e.g., the needle tip 144, the one or more points along the needle shaft 146, etc.) of the needle 106 are determined. The needle-guiding process utilizes the depths determined along the different points of the needle 106 to render, in real-time, at least a portion of a guidance overlay 148 over the ultrasound images for the on-screen guidance of the needle 106 to the target area or anatomical structure thereof, which, in this case, is the foregoing target blood vessel.

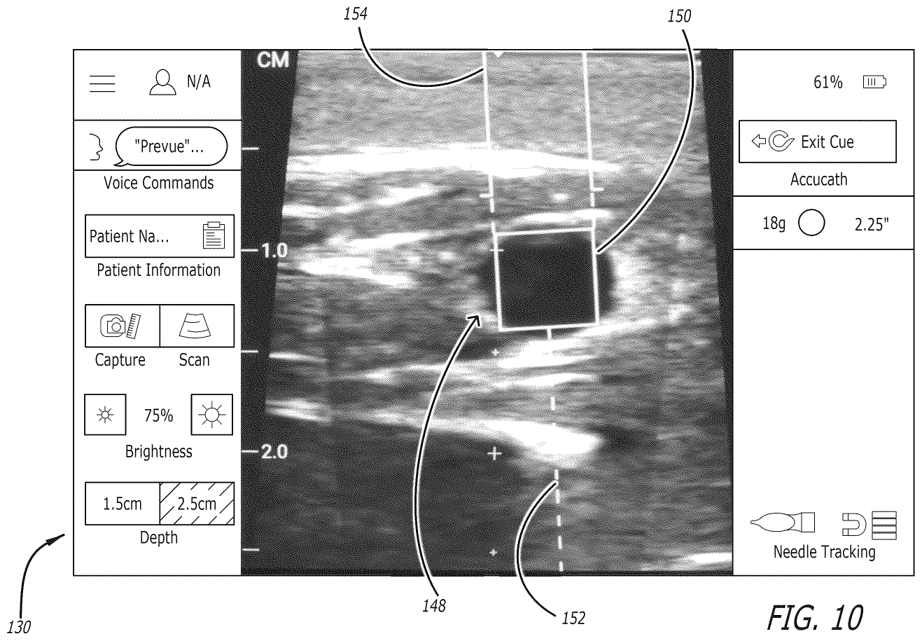

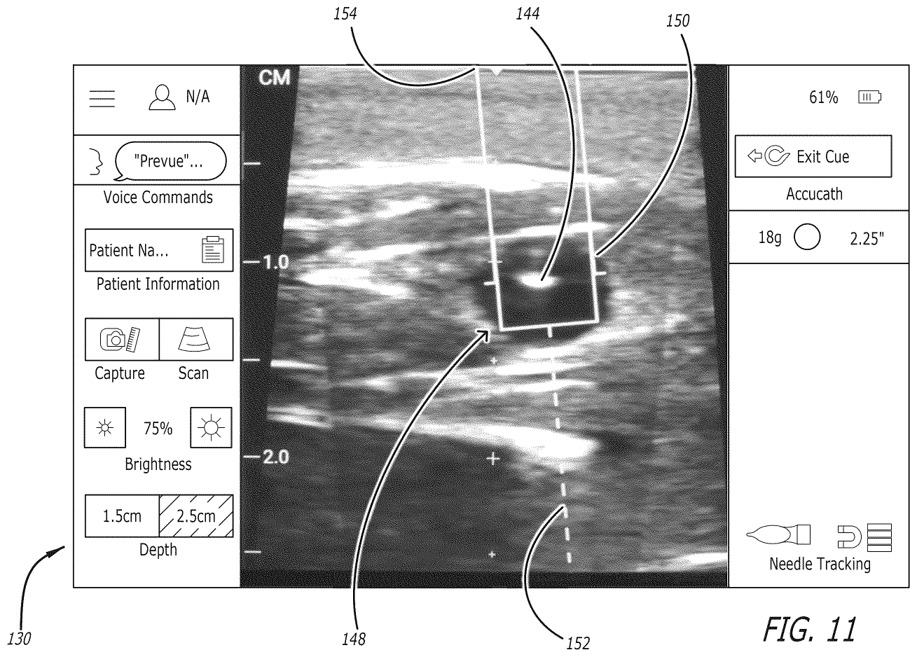

While the guidance overlay 148 can be implemented in any of a number of different ways, the guidance overlay 148 shown in FIGS. 9-11 includes a target box 150, a needle trajectory 154, and a needle-location indicator 154 over the ultrasound images for the on-screen guidance of the needle 106 to the target area or anatomical structure thereof. The target box 150 can be rendered in accordance with one or more local minima in imaging data (e.g., the time of flight and intensity from the echoed ultrasound signals corresponding to the patient) for an instant imaging plane. The needle trajectory 154 can be rendered in real-time from the depths determined along the different points of the needle 106, which different points of the needle 106 necessarily follow a straight line for a straight needle. Lastly, the needle-location indicator 154 can be rendered in real-time from the depth determined for the needle tip 144. As shown in FIG. 9, the needle-location indicator 154 includes orthogonal end ticks indicating the needle tip 144 is a distance of at least 1.0-1.5× the diameter of the target blood vessel away from the target vessel. In FIG. 10, the needle-location indicator 154 likewise indicates the needle tip 144 is at a distance within about 0.5× the diameter of the target blood vessel away from the target vessel. And in FIG. 11, the end ticks of the needle-location indicator 154 along with a needle flash indicate the needle tip 144 is within the target blood vessel.

### Methods

Methods include methods of the ultrasound system 100 itself or methods of using the ultrasound system 100 for needle tracking and guidance.

A method of the ultrasound system 100 for needle tracking and guidance can include instantiating the system process(es) for imaging and needle tracking when executable instructions 124 in the memory 122 of the console 104 are executed by the processor(s) 120 of the console 104. The method can also include generating ultrasound images of a target area or anatomical structure of a patient with the image-generating process of the system process(es). The ultrasound images are generated from source ultrasound signals emitted from the ultrasound-transducing elements of the imaging array 108, which is, again, disposed in the probe head 112 of the ultrasound probe 102, and echoed ultrasound signals from the patient transduced by the ultrasound-transducing elements of the imaging array 108. The method can also include generating needle-tracking data of the needle 106 approaching the target area or anatomical structure of the patient with the needle-tracking process of the system process(es). The needle-tracking data are generated from source ultrasound signals emitted from the ultrasound-transducing elements of the tracking array 110, which is, again, perpendicular to the imaging array 108, and echoed ultrasound signals from the needle 106 transduced by the ultrasound-transducing elements of the tracking array 110. The method can also include providing on-screen guidance of the needle 106 to the target area or anatomical structure thereof with the needle-guiding process of the system process(es).

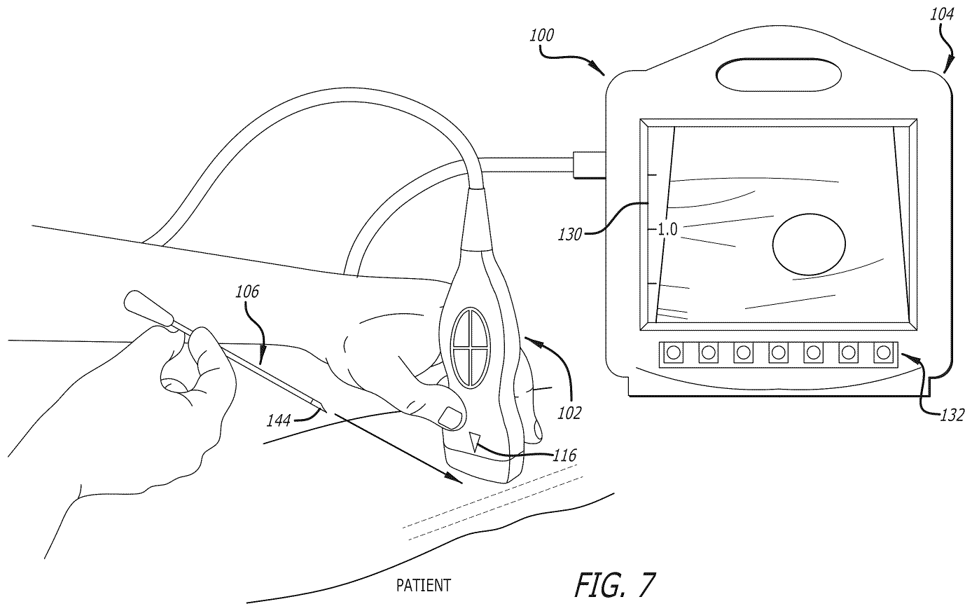

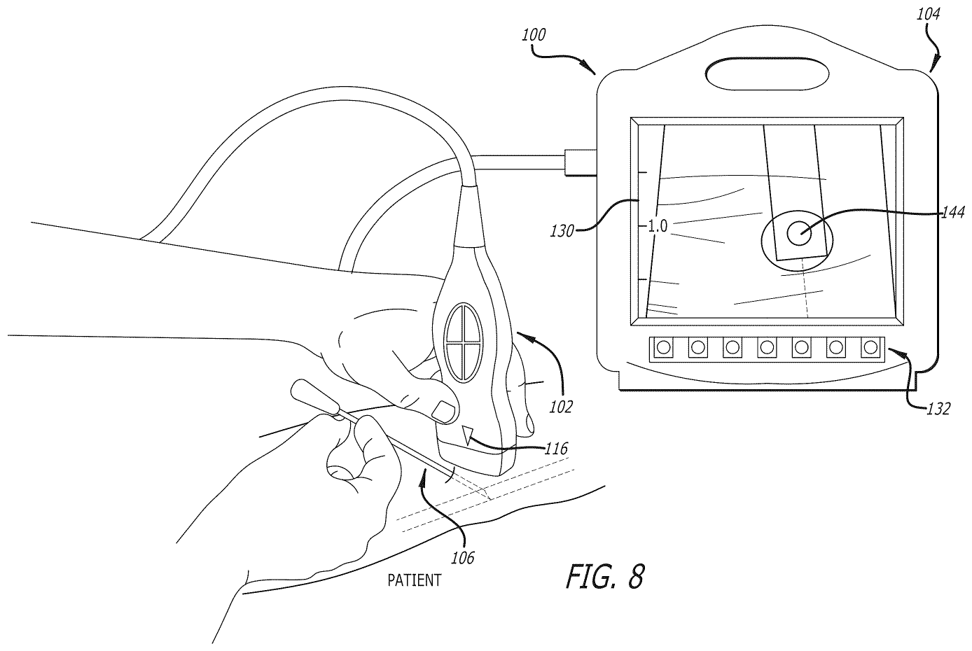

FIGS. 7 and 8 illustrates using the ultrasound system 100 during a percutaneous needle puncture to establishing vascular access in accordance with some embodiments.

A method of using the ultrasound system 100 for needle tracking and guidance can include allowing the ultrasound system 100 to instantiate the system process(es) for imaging and needle tracking set forth in the foregoing method of the ultrasound system 100. Allowing the ultrasound system 100 to instantiate the system process(es) can include switching the ultrasound system 100 on, calling up a desired mode of the ultrasound system 100, or both. The method can also include applying ultrasound gel to a skin surface of a patient over a target area or anatomical structure thereof, which includes the target blood vessel shown, and imaging the target blood vessel with the ultrasound probe 102 as shown in FIG. 7. As further shown in FIG. 7, the method can also include advancing the needle 106 toward the target blood vessel with an approach angle (e.g., between 30° and 45° relative to the skin surface), which approach angle is notably a component of the needle trajectory 154 of the guidance overlay 148. (See FIGS. 9-11.) Notably, the marker 116 is shown on the opposite side of the ultrasound probe 102 from that including the tracking array 110, so the marker 116 indicates the side of the ultrasound probe 102 from which the needle 106 should be inserted into the patient. The method can also include continuing to advance the needle 106 toward the target blood vessel with the approach angle through the resulting percutaneous puncture until the needle tip 144 reaches the anterior wall of target vessel as discerned by the end ticks of the needle-location indicator 154 of the guidance overlay 148. (See FIG. 10.) Lastly, the method can also include reducing the approach angle to that of an insertion angle (e.g., between 20° and 35° relative to the skin surface) and inserting the needle 106 into the target blood vessel until a needle flash is observed in the ultrasound image, the end ticks of the needle-location indicator 154 are aligned with the target blood vessel, or both. (See FIG. 11.)

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. An ultrasound system for needle tracking and guidance, comprising:
an ultrasound probe including:
an imaging array of a plurality of ultrasound-transducing elements disposed in a probe head of the ultrasound probe; and
a linear tracking array of two or more ultrasound-transducing elements perpendicular to the imaging array, the ultrasound-transducing elements of each array of the imaging array and the tracking array oriented to emit source ultrasound signals into a patient and transduce echoed ultrasound signals from both the patient and a needle inserted into the patient; and
a console containing electronic components and circuitry including memory and one or more processors, the memory including executable instructions that instantiate one or more system processes for imaging with the imaging array and needle tracking with the tracking array when executed by the processor(s), and the system processes including:
an image-generating process for generating ultrasound images of a target area or anatomical structure thereof from the echoed ultrasound signals corresponding to the patient;
a needle-tracking process for generating needle-tracking data from the echoed ultrasound signals corresponding to the needle; and
a needle-guiding process for on-screen guidance of the needle to the target area or anatomical structure thereof.

2. The ultrasound system according to claim 1, wherein the tracking array is flush with or recessed into either the probe head or a protrusion laterally extending from the probe head without distally extending past the imaging array.

3. The ultrasound system according to claim 1, wherein the tracking array is angled into the imaging array.

4. The ultrasound system according to claim 1, wherein the ultrasound probe includes a marker on a same side of the ultrasound probe as that including the tracking array, the marker thereby indicating the needle should be inserted into the patient on an opposite side of the ultrasound probe from the marker.

5. The ultrasound system according to claim 1, wherein the ultrasound probe includes a marker on an opposite side of the ultrasound probe than that including the tracking array, the marker thereby indicating the needle should be inserted into the patient on a same side of the ultrasound probe as the marker.

6. The ultrasound system according to claim 1, wherein the ultrasound probe includes a needle-guide mount extending from an opposite side of the ultrasound probe than that including the tracking array.

7. The ultrasound system according to claim 1, wherein the ultrasound system is configured for both A-mode and B-mode pulsed-wave (“PW”) ultrasound.

8. The ultrasound system according to claim 7, wherein the needle-tracking process utilizes the tracking array for A-mode PW ultrasound.

9. The ultrasound system according to claim 8, wherein the ultrasound-transducing elements of the tracking array simultaneously emit the source ultrasound signals and transduce the echoed ultrasound signals for simultaneous depth determinations along different points of the needle.

10. The ultrasound system according to claim 9, wherein the needle-guiding process utilizes the depth determinations along the different points of the needle to render a guidance overlay over the ultrasound images for the on-screen guidance of the needle to the target area or anatomical structure thereof.

11. The ultrasound system according to claim 8, wherein the source ultrasound signals emitted by the ultrasound-transducing elements of the tracking array are frequency modulated, each ultrasound-transducing element of the tracking array emitting its corresponding source ultrasound signals at a unique frequency for independently processing the echoed ultrasound signals transduced by each ultrasound-transducing element of the tracking array in accordance with the unique frequency thereof.

12. The ultrasound system according to claim 8, wherein the source ultrasound signals emitted by the ultrasound-transducing elements of the tracking array are phase modulated, each ultrasound-transducing element of the tracking array emitting its corresponding source ultrasound signals with a unique phase shift for independently processing the echoed ultrasound signals transduced by each ultrasound-transducing element of the tracking array in accordance with the unique phase shift thereof.

13. The ultrasound system according to claim 8, wherein the source ultrasound signals emitted by the ultrasound-transducing elements of the tracking array are pulse encoded, each ultrasound-transducing element of the tracking array emitting its corresponding source ultrasound signals with a unique pulse train for independently processing the echoed ultrasound signals transduced by each ultrasound-transducing element of the tracking array in accordance with the unique pulse train thereof.

14. The ultrasound system according to claim 11, wherein processing the echoed ultrasound signals transduced by each ultrasound-transducing element of the tracking array includes amplitude gating to filter out the echoed ultrasound signals from the patient and isolate the echoed ultrasound signals from the needle, the echoed ultrasound signals from the needle have much greater amplitudes than the echoed ultrasound signals from the patient.

15. The ultrasound system according to claim 7, wherein the image-generating process utilizes the imaging array for B-mode PW ultrasound.

16. The ultrasound system according to claim 15, wherein the source ultrasound signals emitted by the ultrasound-transducing elements of the tracking array being modulated or encoded differentiates them from the source ultrasound signals emitted by the ultrasound-transducing elements of the imaging array and, thus, the echoed ultrasound signals transduced by the ultrasound-transducing elements of the tracking array and the imaging array.

17. An ultrasound probe for needle tracking and guidance, comprising:
an imaging array of a plurality of ultrasound-transducing elements disposed in a probe head of the ultrasound probe; and
a linear tracking array of two or more ultrasound-transducing elements perpendicular to the imaging array, the ultrasound-transducing elements of each array of the imaging array and the tracking array oriented to emit source ultrasound signals into a patient and transduce echoed ultrasound signals from both the patient and a needle inserted into the patient;

18. The ultrasound system according to claim 17, wherein the tracking array is flush with or recessed into either the probe head or a protrusion laterally extending from the probe head so as to not distally extend past the imaging array.

19. The ultrasound system according to claim 17, wherein the tracking array is angled into the imaging array.

20. The ultrasound system according to claim 17, wherein the ultrasound probe includes a marker on a same side of the ultrasound probe as that including the tracking array, the marker thereby indicating the needle should be inserted into the patient on an opposite side of the ultrasound probe from the marker.

21. The ultrasound system according to claim 17, wherein the ultrasound probe includes a marker on an opposite side of the ultrasound probe than that including the tracking array, the marker thereby indicating the needle should be inserted into the patient on a same side of the ultrasound probe as the marker.

22. The ultrasound system according to claim 17, wherein the ultrasound probe includes a needle-guide mount extending from an opposite side of the ultrasound probe than that including the tracking array.

23. A method of an ultrasound system for needle tracking and guidance, comprising:
instantiating one or more system processes for imaging and needle tracking with an ultrasound probe when executable instructions in memory of a console of the ultrasound system are executed by one or more processors of the console;
generating ultrasound images of a target area or anatomical structure of a patient with an image-generating process of the system process(es), the ultrasound images generated from source ultrasound signals emitted from an imaging array of a plurality of ultrasound-transducing elements disposed in a probe head of the ultrasound probe and echoed ultrasound signals from the patient transduced by the ultrasound-transducing elements of the imaging array;
generating needle-tracking data of a needle approaching the target area or anatomical structure of the patient with a needle-tracking process of the system process(es), the needle-tracking data generated from source ultrasound signals emitted from a tracking array of two or more ultrasound-transducing elements perpendicular to the imaging array and echoed ultrasound signals from the needle transduced by the ultrasound-transducing elements of the tracking array;
providing on-screen guidance of the needle to the target area or anatomical structure thereof with a needle-guiding process of the system process(es).
